(19) United States
(12) Patent Application Publication
(10) Pub. No.: US 2016/0223465 A1
(43) Pub. Date: Aug. 4, 2016

(54) METHOD OF DETECTING ANALYTES HAVING A THIOL FUNCTIONAL GROUP

(71) Applicants: AGENCY FOR SCIENCE, TECHNOLOGY AND RESEARCH, Connexis, Singapore (SG); NANYANG TECHNOLOGICAL UNIVERSITY, Singapore (SG)

(72) Inventors: Malini Olivo, Singapore (SG); Kien Voon Kong, Singapore (SG); Weng Kee Leong, Singapore (SG)

(73) Assignees: Agency For Science, Technology and Research, Singapore (SG); Nanyang Technological University, Singapore (SG)

(21) Appl. No.: 14/917,781

(22) PCT Filed: Sep. 18, 2014
(86) PCT No.: PCT/SG2014/000448

§ 371 Date: Mar. 9, 2016

(30) Foreign Application Priority Data
Sep. 18, 2013 (SG) 201307050-3

(51) Int. Cl. G01N 21/65; G01N 33/50; G01N 33/52; G01N 21/78

(52) U.S. Cl. CPC G01N 21/658; G01N 21/78; G01N 33/5005; G01N 33/52

(57) ABSTRACT

Method of detecting one or more analytes having a thiol functional group is provided. The method includes contacting one or more analytes with at least one metal carbonyl cluster compound; and detecting changes in optical properties of the at least one metal carbonyl cluster compound as an indication of the presence of the one or more analytes having a thiol functional group.

### CROSS-REFERENCE TO RELATED APPLICATION

This application claims the benefit of priority of Singapore patent application No. 201307050-3 filed on 18 Sep. 2013, the content of which is incorporated herein by reference in its entirety for all purposes.

### TECHNICAL FIELD

The invention refers to a method of detecting analytes having a thiol functional group.

### BACKGROUND

Surface-enhanced Raman scattering (SERS) is a powerful spectroscopy technique discovered several decades ago and has been well studied. It has dramatically improved the inherent low sensitivity of Raman spectroscopy in which Raman signals of molecules on nanostructured surfaces may be enhanced by several orders of magnitude (typically 10⁶ to 10¹⁴), due to the strong surface plasmon resonance of the nanostructured surface.

While there have been reports that utilized UV-vis, electrochemical, fluorescence, colorimetric techniques for thiol detection, a probe with SERS and colorimetric detection for dual-modal sensing remains unexplored. There is also no report of a SERS probe for analytes containing a thiol functional group. One reason is that, functional groups which are used to react with thiols, such as maleimide, do not have high Raman cross-sections and their Raman signals, which lie in the region of 400 cm⁻¹ to 1800 cm⁻¹ may be easily interfered by signals from biomolecules present in the cell.

In view of the above, there exists a need for an improved method of detecting analytes that contain a thiol functional group.

### SUMMARY

In a first aspect, the invention refers to a method of detecting one or more analytes having a thiol functional group. The method comprises
a. contacting one or more analytes with at least one metal carbonyl cluster compound; and
b. detecting changes in optical properties of the at least one metal carbonyl cluster compound as an indication of the presence of the one or more analytes having a thiol functional group.

### BRIEF DESCRIPTION OF THE DRAWINGS

The invention will be better understood with reference to the detailed description when considered in conjunction with the non-limiting examples and the accompanying drawings, in which:

FIG. 1A is a schematic diagram showing sample preparation for SERS measurement with bi-metallic film over nanosphere (BMFON). FIG. 1B is a scanning electron microscopy (SEM) image, and FIG. 1C is a photograph of BMFON. FIG. 1D is a graph showing Raman spectrum of Os₃(CO)₁₀(NCMe)₂ (Me represents methyl group) on glass slide and BMFON. Scale bar in FIG. 1B and FIG. 1C represents 1 μm and 0.25 cm respectively.

FIG. 2 is a graph showing SERS spectra of (a) Os₃(CO)₁₀(NCMe)₂, (b) Os₃(CO)₁₀(NCMe)₂+FMOC-S-trityl-L-cysteine, (c) Os₃(CO)₁₀(NCMe)₂+N-(tert-butoxycarbonyl)-L-cysteine methyl ester, (d) Os₃(CO)₁₀(NCMe)₂+N-acetyl-L-cysteine, (e) Os₃(CO)₁₀(NCMe)₂+cysteine, and (f) Os₃(CO)₁₀(NCMe)₂+GSH.

FIG. 3 is a graph showing Raman spectra of Os₃(CO)₁₀(NCMe)₂ after incubation with different amino acids of alanine, aspartic acid, glutamic acid, glycine, glutamine, phenylalanine, tryptophan, tyrosine, leucine, serine, proline, isoleucine, proline, glutathione disulfide (GSSG), and glutathione (GSH); and bovine serum albumin (BSA).

FIG. 4 is a graph depicting transformation of Os₃(CO)₁₀(NCMe)₂ to Os₃(CO)₁₀(μ-H)(μ-SR) species after incubation with GSH. Two species, Os₃(CO)₁₀(μ-H)(μSR) and Os₃(CO)₁₀(μ-H)(μ-OOCR) were detected in high concentrations of Os₃(CO)₁₀(NCMe)₂.

FIG. 5 is a graph showing calibration curve of Os₃(CO)₁₀(NCMe)₂ as it reacts with different concentrations of GSH. Y-axis shows intensity of the 2111 cm⁻¹ SERS peak attributed to Os₃(CO)₁₀(μ-H)(μ-SR), where R is GSH.

FIG. 6 is a graph showing SERS spectrum of cell incubated with Os₃(CO)₁₀(NCMe)₂ (a) with, and (b) without subsequent introduction of gold nanoparticles. Inset: enlarged 2000 cm⁻¹ to 2100 cm⁻¹ region.

FIG. 7A to 7J shows bright field and SERS mapping images of OSCC treated with Os₃(CO)₁₀(NCMe)₂. Images of OSCC cells treated with Os₃(CO)₁₀(NCMe)₂ prior to incubation with gold nanoparticles (60 nm) (A to E). Images of OSCC cells treated with Os₃(CO)₁₀(NCMe)₂ without gold nanoparticles (F to J). All SERS mapping images of CO (2111 cm⁻¹) was scanned at an interval of 1 mm (785 nm excitation). Scale bar in FIGS. 7A and 7F represents 20 μm; scale bar in FIG. 7B to 7E, and FIG. 7G to 7J represents 10 μm.

FIG. 8 is a schematic diagram showing general reactivity of Os₃(CO)₁₀(μ-H)₂ with thiol.

FIG. 9 is a schematic diagram showing preparation of Os₃(CO)₁₀(μ-H)₂.

FIG. 10 is a schematic diagram showing SERS and colorimetric thiol detection using Os₃(CO)₁₀(μ-H)₂.

FIG. 11A to 11C show (A) photograph; (B) UV-vis spectrum; and (C) SERS spectrum of Os₃(CO)₁₀(μ-H)₂ (1 mM) before and after adding cysteine (1 mM).

FIG. 12A to 12D show (A) molecular structure of various protected cysteines of FMOC-S-trityl-L-cysteine, N-(tert-butoxycarbonyl)-L-cysteine methyl ester, and N-acetyl-L-cysteine; (B) photograph; and (C) SERS spectrum of Os₃(CO)₁₀(μ-H)₂, and Os₃(CO)₁₀(μ-H)₂ added respectively with FMOC-s-trityl-L-cysteine, N-(tert-butoxycarbonyl)-L-cysteine methyl ester, and N-acetyl-L-cysteine. FIG. 12D depicts an enlarged 2100 cm⁻¹ to 2150 cm⁻¹ region of FIG. 12C.

FIGS. 13A and 13B show (A) SERS spectra, and (B) photograph of Os₃(CO)₁₀(μ-H)₂ treated with other natural amino acids of glutamic acid (Glu), aspartic acid (Asp), glutamine (Gln), phenylalanine (Phe), tryptophan (Trp), leucine (Leu), serine (Ser), proline (Pro), alanine (Ala), and glutathione (GSH).

FIG. 14 shows reaction of Os₃(CO)₁₀(μ-H)₂ with 20 different amino acids of alanine (A10), arginine (A9), aspartic acid (A8), asparagine (A7), glutamine (A6), glutamic acid (A5), glycine (A4), isoleucine (A3), phenylalanine (A2), GSH (A1), tryptophan (B10), tyrosine (B9), valine (B8), lysine (B7), histidine (B6), leucine (B5), serine (B4), proline (B3), threonine (B2), and cysteine (B1).

FIG. 15 shows (A) photograph depicting color change before and after addition of Os₃(CO)₁₀(μ-H)₂ to GSH at various concentrations of 1 mM, 0.75 mM, 0.5 mM, 0.25 mM, 0.1 mM GSH; GSSG, and water. (B) is a plot at different concentrations of GSH. (C) are photographs showing color change in clear and bulk urine before and after addition of GSH. (D) is a SERS spectrum of Os₃(CO)₁₀(μ-H)₂ after incubation with clinical urine.

### DETAILED DESCRIPTION

Various embodiments refer to a dual mode method of detecting analytes having a thiol functional group. In some embodiments, a metal carbonyl cluster compound, such as a triosmium carbonyl cluster, is used as a probe for recognition and quantification of thiol containing biomolecules.

The method allows detection to be carried out under a colorimetric mode and/or SERS mode. Excellent selectivity towards thiol functionality, with very fast detection time in order of seconds, has been demonstrated. It has been shown that analytes having a thiol functional group may be detected in two seconds. Advantageously, by observing a distinct color change from purple to yellow, which is discernible to the naked eye, analytes containing a thiol functional group may be detected quickly and conveniently without using expensive, sophisticated instruments.

The colorimetric mode may be complemented by a SERS mode, which is highly sensitive. Methods according to embodiments disclosed herein involves use of a metal carbonyl cluster compound, which is able to provide a unique SERS signal at a mid-IR region of 1800 cm⁻¹ to 2200 cm⁻¹ from CO stretching vibrations. The CO stretching vibration frequency is strongly correlated with species formed on reaction of the metal carbonyl cluster compound with thiol, and these vibrations occur in a region of the mid-IR, which is free from interference by biomolecules. In addition, this technique provides quantification and high sensitivity even with clinical samples. Only a small volume of analyte is required, and components of the protocol may be prepared easily.

With the above in mind, various embodiments relate in a first aspect to a method of detecting one or more analytes having a thiol functional group. The terms “thiol group” and “mercapto group” are used interchangeably herein and both relate to the —SH group.

The term “detecting” as used herein refers to a method of verifying the presence of a given molecule, and includes in vitro as well as in vivo detection. The detection may also be quantitative, such as correlating the detected signal with amount of analytes present. The method of detecting one or more analytes having a thiol functional group may also be a multiplex method for detecting more than one analyte, such as two or more different analytes.

The method comprises contacting one or more analytes with at least one metal carbonyl cluster compound, and detecting changes in optical properties of the at least one metal carbonyl cluster compound as an indication of the presence of the one or more analytes having a thiol functional group.

As used herein, the term “metal carbonyl cluster compound” refers to metal cluster compounds comprising carbon monoxide in complex combination with metal atoms, wherein the metal atoms in the metal carbonyl cluster are held together entirely or at least substantially by bonds between metal atoms.

The carbonyl ligands and/or other ligands in the metal carbonyl cluster compound may be bonded to some or all of the metal atoms to form a complex. In some embodiments, a carbonyl ligand is bonded to two metal atoms to form a bridge between the two metal atoms. Other suitable bridging groups may include, for example, phosphine, arsine, and mercapto groups.

The at least one metal carbonyl cluster compound may have general formula (I)

M₃(CO)ₓL₁₂₋ₓ (I)

wherein M at each occurrence denotes a metal selected from Group 6 to Group 11 of the Periodic Table of Elements; x is an integer from 10 to 12; and each L is independently a ligand having a dissociation constant that is at least 1×10⁻³ s⁻¹.

As used herein, the term “dissociation constant” refers to equilibrium constant that measures propensity of a larger entity to separate or dissociate reversibly into smaller components. Dissociation constant may, for example, by determined by the following using Os₃(CO)₁₀(μ-H)(μ-SR) as an example.

Immediately after mixing the reactants to form a reaction mixture, a nuclear magnetic resonance spectroscopy (NMR) spectroscopy may be carried out on the reaction mixture, where the NMR spectra show presence of resonances belonging to starting material Os₃(CO)₁₀(NC—CH₃)₂, intermediate Os₃(CO)₁₀(HSR) and final product Os₃(CO)₁₀(μ-H)(μ-SR). Concentrations of the three species may be evaluated from integrals of the signals at 2.72 ppm for CH₃CN in Os₃(CO)₁₀(NC—CH₃)₂ and of the agostic hydrogen of the intermediate Os₃(CO)₁₀(HSR) and hydrid resonances of the final product Os₃(CO)₁₀(μ-H)(μ-SR). Time dependence of the concentrations for the three species may be used to calculate dissociate constant.

In the context of the present application and with reference to formula (I), dissociation constant may be used as a measure to describe affinity between ligand L with metal atom M. Generally, a higher dissociation constant means that the ligand is more loosely bound to the metal atom, and has a greater tendency to separate, or dissociates easily from the metal atom.

As mentioned above, each L in formula (I) may be independently a ligand having a dissociation constant that is at least 1×10⁻³ s⁻¹. For example, the ligand may have a dissociation constant that is at least 2×10⁻³ s⁻¹, at least 4×10⁻³ s⁻¹, at least 6×10⁻³ s⁻¹, at least 8×10⁻³ s⁻¹, at least 1×10⁻² s⁻¹, or in the range of about 1×10⁻³ s⁻¹ to about 0.1 s⁻¹, such as about 1×10⁻³ s⁻¹ to about 9×10⁻² s⁻¹, about 1×10⁻³ s⁻¹ to about 5×10⁻² s⁻¹, about 1×10⁻³ s⁻¹ to about 2×10⁻² s′¹, about 1×10⁻³ s⁻¹ to about 9×10⁻³ s⁻¹, about 1×10⁻³ s⁻¹ to about 5×10⁻² s⁻¹, about 1×10⁻² to about 9×10⁻² s⁻¹, or about 1×10⁻² to about 5×10⁻² s⁻¹.

In exemplified embodiments disclosed herein, M is osmium and L is acetonitrile. Dissociation constant of osmium-acetonitrile is (1.52±0.1)×10⁻² s⁻¹. This value of dissociation constant provides acetonitrile ligands with ability to dissociate rapidly, which undergo oxidation addition reaction with a thiol group, and which form basis of a method of detecting analytes comprising a thiol group as disclosed herein.

In various embodiments, each L is independently a ligand having a dissociation constant that is higher than that of a thiol ligand. In having a dissociation constant that is higher than that of a thiol ligand, this means that the ligand has a greater tendency to dissociate from the metal atom in the metal carbonyl cluster compound as compared to a thiol functional group. This allows formation of a thiolated bridged cluster, and in turn a color change due to changes in CO stretching vibration. Examples of ligands having a dissociation constant that is higher than that of a thiol ligand may include, but are not limited to, —H, —NC, —CH₃, and —NC—(CH₂)ₙ—CH₃, wherein n is 0 or an integer from 1 to 10.

In specific embodiments, each L is independently selected from the group consisting of —H, and —NC—(CH₂)ₙ—CH₃, wherein n is 0 or an integer from 1 to 10. CO in formula (I) denotes a carbonyl ligand.

In various embodiments, M is independently selected from the group consisting of chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), and gold (Au). In some embodiments, M is independently selected from the group consisting of Fe, Ru, and Os. In specific embodiments, M is Os.

The metal carbonyl cluster compound may comprise more than one metal. For example, M in the general formula M₃(CO)ₓL₁₂₋ₓ, mentioned above may be represented by (Mₐ)₂M_{b}, Mₐ(M_{b})₂, (M_{b})₂M_{c}, M_{b}(M_{c})₂, (Mₐ)₂M_{c}, Mₐ(M_{c})₂, or MₐM_{b}M_{c}, where Mₐ, M_{b} and M_{c} denote different metals. In such embodiments, the metal carbonyl compound may have general formula MₐM_{b}M_{c}(CO)ₓL₁₂₋ₓ, wherein Mₐ, M_{b}, and M_{c} denote different metals, and CO, L, and x having the same definitions as that mentioned above.

x is an integer from 10 to 12. For example, x may be 10, 11, or 12. In specific embodiments, x is 10.

L denotes a ligand in the metal carbonyl cluster compound. L at each occurrence may be the same or different. Each L is independently selected from the group consisting of —H, and —NC—(CH₂)ₙ—CH₃. n is 0 or an integer from 1 to 10, such as 1, 2, 3, 4, 5, 6, 7, 8, 9 or 10.

In various embodiments, n is 0.

In various embodiments, L includes —H and —NC—CH₃.

In various embodiments, the metal carbonyl cluster compound is selected from the group consisting of Os₃(CO)₁₀(μ-H)₂, Os₃(CO)₁₀(NC—CH₃)₂, and combinations thereof. In the formula, the symbol “μ” is used to denote a bridging atom. Hence, in the compound Os₃(CO)₁₀(μ-H)₂, H is a bridging atom. In specific embodiments, the metal carbonyl cluster compound is Os₃(CO)₁₀(μ-H)₂.

The terms “contacting” or “incubating” as used interchangeably herein refer generally to providing access of one component, reagent, analyte or sample to another.

In various embodiments, contacting one or more analytes with at least one metal carbonyl cluster compound may include incubating the one or more analytes with the at least one metal carbonyl cluster compound. The one or more analytes and the at least one metal carbonyl cluster compound may be incubated for a suitable time that allows interaction between the analyte and the metal carbonyl cluster compound to take place. A suitable amount of time may be dependent on reaction conditions, such as type and amount of analyte and metal carbonyl cluster compound, and temperature. A person skilled in the art would be able to determine the appropriate amount of time for any interaction that may take place to occur.

Typically, incubating the one or more analytes with the at least one metal carbonyl cluster compound may take place for a period of time in the order of hours, and be carried out at ambient temperature, which generally refers to a temperature of between about 20° C. to about 40° C. In various embodiments, incubating the one or more analytes with the at least one metal carbonyl cluster compound is carried out for about 2 hours at about 25° C.

In some embodiments, contacting one or more analytes with at least one metal carbonyl cluster compound may include incubating the one or more analytes and the at least one metal carbonyl cluster compound with a metallic nanoparticle.

One or a plurality of metallic nanoparticles may be present. The term “plurality” as used herein means more than one, such as at least 2, 20, 50, 100, 1000, 10000, 100000, 1000000, 10000000, or even more.

The metallic nanoparticle may be coated with or consists of a SERS-active material. Examples of a SERS-active material include, but are not limited to, noble metals such as silver, palladium, gold, platinum, iridium, osmium, rhodium, ruthenium; copper, aluminum, or alloys thereof.

For example, the metallic nanoparticle may be formed entirely from a SERS metal, and may for example, consist of a metal selected from the group consisting of a noble metal, copper, aluminum, and alloys thereof. In various embodiments, the metallic nanoparticle is coated with or consists of gold, silver, or alloys thereof. In specific embodiments, the metallic nanoparticle is coated with or consists of gold.

As another example, the metallic nanoparticle may be formed from a non-SERS active material, such as plastic, ceramics, composites, glass or organic polymers, and coated with a SERS metal such as that mentioned above.

Size of the metallic nanoparticle may be characterized by its diameter. The term “diameter” as used herein refers to the maximal length of a straight line segment passing through the center of a figure and terminating at the periphery. In embodiments where a plurality of metallic nanoparticles is present, size of the metallic nanoparticles may be characterized by their mean diameter. The term “mean diameter” refers to an average diameter of the nanoparticles, and may be calculated by dividing sum of the diameter of each nanoparticle by the total number of nanoparticles.

In various embodiments, the metallic nanoparticle or nanoparticles have a diameter or a mean diameter of about 30 nm to about 80 nm, such as about 40 nm to about 80 nm, about 50 nm to about 80 nm, about 60 nm to about 80 nm, about 30 nm to about 70 nm, about 30 nm to about 60 nm, about 50 nm to about 70 nm, or about 60 nm.

Where a plurality of metallic nanoparticles is present, the nanoparticles may be monodisperse. The term “monodisperse” refers to nanoparticles having a substantially uniform size and shape. In some embodiments, the standard deviation of diameter distribution of the metallic nanoparticles is equal to or less than 20% of the mean diameter value, such as equal to or less than 15%, 10%, 5% or 3% of the mean diameter value. In some embodiments, the diameter of the metallic nanoparticles is essentially the same.

Method disclosed herein includes detecting changes in optical properties of the at least one metal carbonyl cluster compound as an indication of the presence of the one or more analytes having a thiol functional group.

In various embodiments, detecting changes in optical properties of the at least one metal carbonyl cluster compound is carried out with a naked eye and/or a spectrometer.

If the change of the optical properties is visible in the light wave range visible to humans, it is possible to detect changes in optical properties of the at least one metal carbonyl cluster compound with the naked eye. Using this method, detection may be carried out in a simple and fast manner by observing color change in solution without using analytical instruments, which is advantageous as the analytical instruments may be costly.

In some embodiments, detecting changes in optical properties of the at least one metal carbonyl cluster compound is carried out with a spectrometer. Detecting changes in optical properties with a spectrometer may include detecting changes in surface enhanced Raman signal from the at least one metal carbonyl cluster compound.

In various embodiments, detecting changes in surface enhanced Raman signal from the at least one metal carbonyl cluster compound comprises detecting changes in pattern and/or intensity of surface enhanced Raman signal in the region of 1800 cm⁻¹ to 2200 cm⁻¹.

Advantageously, the at least one metal carbonyl cluster compound is able to provide a unique SERS signal at a mid-IR region of 1800 cm⁻¹ to 2200 cm⁻¹ from CO stretching vibrations, thereby avoiding interference with signals emitted by biomolecules which are in the 800 cm⁻¹ to 1800 cm⁻¹ region. This allows identification of biomolecules without the need to decouple signals emitted from the metal carbonyl cluster compound. This attribute may be used to provide a more complex spectrum for multiplex detection.

Besides detecting presence of one or more analytes having a thiol functional group, the method disclosed herein may also be used to determine amount of the one or more analytes. In various embodiments, amount of the one or more analytes having a thiol functional group is correlated with surface enhanced Raman signal from the at least one metal carbonyl cluster compound.

In some embodiments, the one or more analytes having a thiol functional group is contained in a sample and the detection is in vitro.

The term “sample”, as used herein, refers to an aliquot of material, frequently biological matrices, an aqueous solution or an aqueous suspension derived from biological material. Samples to be assayed for the presence of an analyte include, for example, cells, tissues, homogenates, lysates, extracts, and purified or partially purified proteins and other biological molecules and mixtures thereof.

Non-limiting examples of samples include human and animal body fluids such as whole blood, serum, plasma, cerebrospinal fluid, sputum, bronchial washing, bronchial aspirates, urine, semen, lymph fluids and various external secretions of the respiratory, intestinal and genitourinary tracts, tears, saliva, milk, white blood cells, myelomas and the like; biological fluids such as cell culture supernatants; tissue specimens which may or may not be fixed; and cell specimens which may or may not be fixed. The samples used may vary based on the assay format and the nature of the tissues, cells, extracts or other materials, especially biological materials, to be assayed. Methods for preparing protein extracts from cells or samples are well known in the art and can be readily adapted in order to obtain a sample that may be used in the method disclosed herein.

The one or more analytes having a thiol functional group may be detected in a body fluid comprising the analyte. In specific embodiments, the body fluid is selected from the group consisting of plasma, serum, blood, lymph, liquor and urine. Detection in a body fluid may also be in vivo, i.e. without first collecting a sample.

Method according to embodiments disclosed herein may form the basis of detection in biosensors, such as SERS-based biomarker assays for clinical diagnosis and assay for use in laboratory research.

For example, method disclosed herein may be used to determine concentration of thiols in patients' clear and bulk urine samples. Deviations in the amount of urinary thiol excretion may be a sign of health disorders, where elevated level of urinary excretion of thiols has been associated with patients with inflammation, myocardial infarction, cancers and autoimmune diseases like rheumatoid arthritis. On the other hand, patients with proteinuria showed significantly decrease level of urinary protein thiols as compared to healthy controls. As such, urine samples are able to provide valuable information for instant disease diagnosis, where serum samples are not affordable and accessible.

The method may also be used to determine concentration of protein in low sample volumes, which may be carried out using only a short incubation time in the order of seconds.

Hereinafter, the present invention will be described more fully with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art. In the drawings, lengths and sizes of layers and regions may be exaggerated for clarity.

As used herein, the term “and/or” includes any and all combinations of one or more of the associated listed items. The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

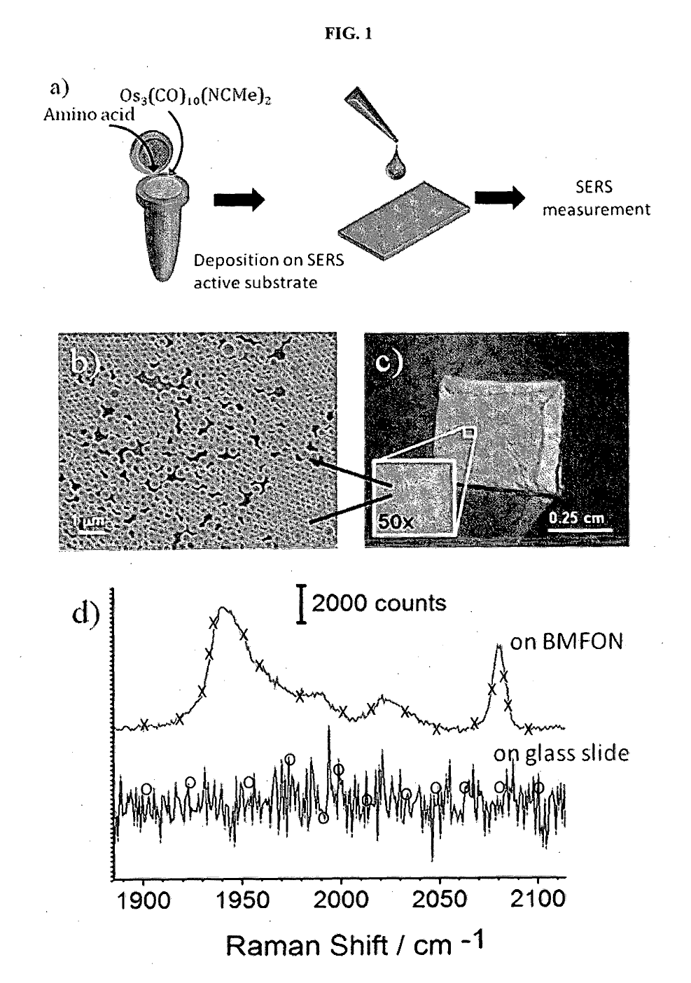

Os₃(CO)₁₀(NCMe)₂ is a reactive organometallic compound with high affinity towards thiol (SH) functionality as compared to carboxyl (COOH) and amide (NH₂) functionalities. The labile acetonitrile ligands can dissociate rapidly [osmium-acetonitrile dissociation rate constant is (1.52±0.1)×10⁻² s⁻¹] and undergo oxidation addition reaction with SH group. These properties render Os₃(CO)₁₀(NCMe)₂ useful in thiol detection. As reaction with biomolecules would significantly alter its metal carbonyl CO stretching vibrations, the weak Raman signals of these vibrations may be significantly enhanced if the process was monitored on a SERS-active substrate (FIG. 1A).

As disclosed herein, inclusion of both colorimetric mode and SERS mode combine the high sensitivity of SERS technique with convenience and visual appeal of a colorimetric technique for a more robust detection. In various embodiments, triosmium carbonyl cluster 1, Os₃(CO)₁₀(μ-H)₂, have been prepared for thiol detection via distinct changes in the color and SERS spectrum. It exhibits strong CO stretching vibrations at the mid-IR (1800-2200 cm⁻¹) which is relatively devoid of interference from other functional groups. Cluster 1 is electronically unsaturated (46 electrons) with a distinct purple color. It demonstrates a higher reactivity towards thiol (—SH) functional group over other groups, forming the μ,κS-thiolate bridged cluster (48 electrons). Formation of the thiolate bridged cluster resulted in changes in the CO stretching vibration and a color change from purple to yellow. To the inventors' knowledge, this is the first report of a probe that exhibits SERS and colorimetric dual-modal detection of thiol molecules.

### Example 1

### General Procedure

All manipulations for chemical synthesis were carried out using standard Schlenk techniques under an argon or nitrogen atmosphere. The triosmium carbonyl cluster Os₃(CO)₁₀(μ-H)₂, was prepared using the following procedure. Briefly, Os₃(CO)₁₂ in dichloromethane is reacted with trimethylamine oxide in methyl alcohol in the presence of a little methyl cyanide at room temperature to yield almost quantitatively the methyl cyanide complex [OS₃(CO)₁₁(NCMe)]. The reaction of [OS₃(CO)₁₁(NCMe)] with H₂ gives Os₃(CO)₁₀(μ-H)₂. Os₃(CO)₁₂ was purchased from Oxkem; all other chemicals were purchased from other commercial sources and used as supplied. UV-vis spectra were recorded using a Beckman Coulter DU 730 spectrometer.

Infrared spectra were recorded on a Bruker Alpha FT-IR spectrometer. Solution spectrum were recorded in DCM solution, in a solution IR cell with NaCl windows and a path length of 0.1 mm, at a resolution of 2 cm⁻¹. HRMS were recorded in ESI mode on a Waters UPLC-Q-Tof MS mass spectrometer. The spectral measurements were carried out using a Renishaw InVia Raman (UK) microscope with a Peltier cooled CCD detector and an excitation wavelength at 633 nm, where the laser beam is directed to the sample through a 50× objective lens, which was used to excite the sample and also to collect the return Raman signal. All Raman spectra were processed with WiRE3.0 software. The maximum laser power at the sample was measured to be 6.2 mW and the exposure time was set at 10 s throughout the measurements. Prior to each measurement, the instrument was calibrated with a silicon standard whose Raman peak is centered at 520 cm⁻¹.

### Example 2

### Detection of Thiol Containing Biomolecules

Freshly prepared 5 mM solutions of Os₃(CO)₁₀(NCMe)₂ in acetonitrile (200 μL) was mixed with amino acid aqueous solution (200 μL) and incubated for 1 hour. 20 μL of mixed solution was dropped on BMFON and dry in air for 15 min.

### Example 3

### SERS Mapping Experiments in OSCC Cells

SERS mapping experiments were performed with a Renishaw InVia Raman microscope system with a laser beam directed to the sample through a 50× objective lens, and a Peltier cooled CCD detector. OSCC (epidermoid carcinoma) cells (from ATCC) were seeded in an 8-well glass slide at a density of 10⁶ cells/mL, together with Dulbecco Modified Eagle's Medium (DMEM, Gibco) containing 10% fetal bovine serum and penicillin streptomycin (Gibco). All cultures were maintained at 37° C. with 5% CO₂.

After incubation with Os₃(CO)₁₀(NCMe)₂ (200 μM) for 2 h at 25° C., followed by rinsing with media (×1), the samples were subsequently incubate with 60 nm of gold nanoparticles (2.6×10¹⁰ particles/mL, BBInternational UK) for another one hour, followed by rinsing with media.

The samples were excited with a 785 nm laser with a focal spot of 1 μm and 300 mW power, and the mapping measurements at 2111 cm⁻¹ were carried out as raster scans in 1 μm steps over the specified area (approx. 30×30 μm²) with 1 s as the integration time per step.

The cells were subsequently mounted with Vectashield fluorescent mounting medium for dark-field microscopy experiments, and visualized using an enhanced dark field (EDF) illumination system (CytoViva) attached to a Nikon Eclipse 80 i microscope. The system consisted of a CytoViva 150 dark-field condenser that was put in place of the original condenser of the microscope, and attached via a fiber optic light guide to a Solarc 24 W metal halide light source. Images were taken under a 100× oil objective lens with an iris.

### Example 4

### Detection of Thiol Containing Biomolecules

200 μL of freshly prepared solutions of Os₃(CO)₁₀(μ-H)₂ in ethanol (1 mM) was mixed with amino acid aqueous solution (200 μL) and incubated for 10 secs. 20 μL of mixed solution was drop on BMFON and dry in air for 15 min. The Raman spectral measurements were then carried out.

### Example 5

### Detection of Thiol in Clinical Urine Sample

In this experiment, human urine sample was provided by Dr. Weber Lau in Singapore General Hospital. 200 μL of Os₃(CO)₁₀(μ-H)₂ in ethanol (1 mM) was added to urine samples (200 μL). The final urine solution was agitated. SERS measurement of urine was taken on BMFON. For the thiol detection with Ellman's reagent, the experiment was performed according to the following.

Briefly, DTNB stock solution and Tris dilution buffer were prepared. For DTNB stock solution, 50 mM sodium acetate and 2 mM DTNB in H₂O were mixed; for Tris solution, 1 M Tris/pH 8.0 was used. A standard SH (acetyl Cysteine) calibration curve was prepared, starting with 10 mM concentration. 50 μL of the DTNB solution, 100 μL Tris solution, and 840 μL water were mixed carefully using a pipette. A 10 μl sample solution was added to 990 μl DTNB mixture. The mixture was mixed well and incubated for 5 min at room temperature. The optical absorbance was measured at 412 nm.

### Example 6

### Results and Discussion for Detection of Thiol

In this study, reaction conditions have been optimized by using 1:1 acetonitrile and water to give maximum solubility to Os₃(CO)₁₀(NCMe)₂ and amino acids. Moreover, Os₃(CO)₁₀(NCMe)₂ is stable in acetonitrile and can be stored.

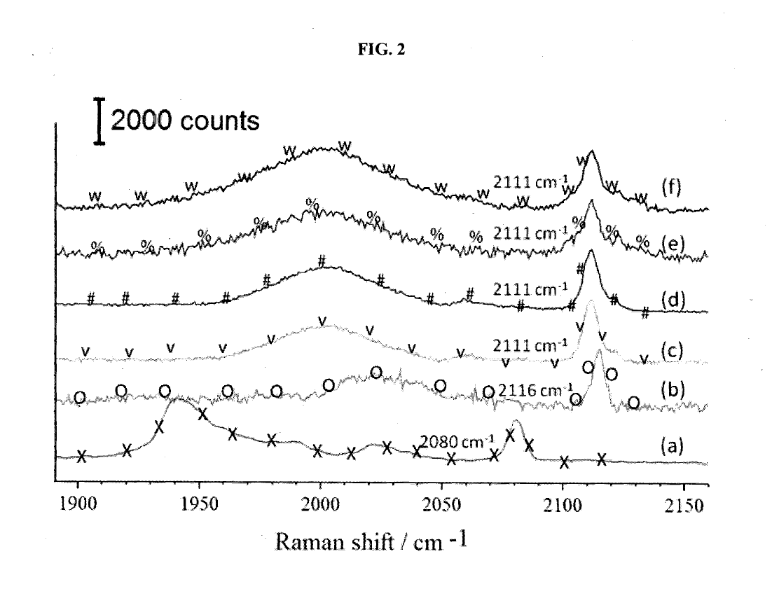

FIG. 2 is a graph showing SERS spectra of (a) Os₃(CO)₁₀(NCMe)₂, (b) Os₃(CO)₁₀(NCMe)₂+FMOC-S-trityl-L-cysteine, (c) Os₃(CO)₁₀(NCMe)₂+N-(tert-butoxycarbonyl)-L-cysteine methyl ester, (d) Os₃(CO)₁₀(NCMe)₂+N-acetyl-L-cysteine, (e) Os₃(CO)₁₀(NCMe)₂+cysteine, and (f) Os₃(CO)₁₀(NCMe)₂+GSH.

Structure of the respective compounds are shown below:

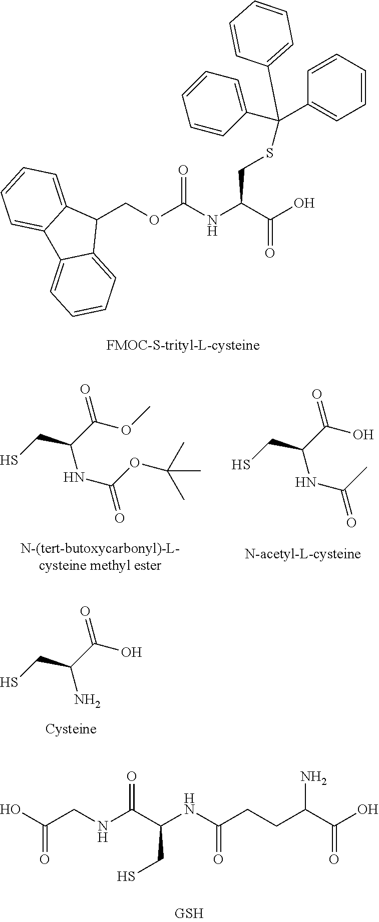

After 1:1 ratio mixing of Os₃(CO)₁₀(NCMe)₂ with GSH, cysteine and protected cysteines, it gives an altered spectrum (FIG. 2). The initial SERS peak of Os₃(CO)₁₀(NCMe)₂ at 2,080 cm⁻¹ is altered to 2,111 cm⁻¹ for GSH, cysteine and SH non-protected cysteine. The appearance of peak at 2111 cm⁻¹ is due to formation of Os₃(μ-H)(CO)₁₀(μ-SR) species.

In the SH protected cysteine, a new peak at 2,116 cm⁻¹ was observed. This peak was also observed in other SH free amino acids such as alanine (FIG. 3). The appearance of 2,116 cm⁻¹ was certainly attributed by the reaction of COOH group in amino acid with Os₃(CO)₁₀(NCMe)₂. It shows that Os₃(CO)₁₀(NCMe)₂ interacts with SH and COOH group over other competing functional groups. Furthermore, Os₃(CO)₁₀(NCMe)₂ could also react with thiol-containing proteins such as BSA (bovine serum albumin) in 1:1 ratio giving rise to 2,111 cm⁻¹ peak.

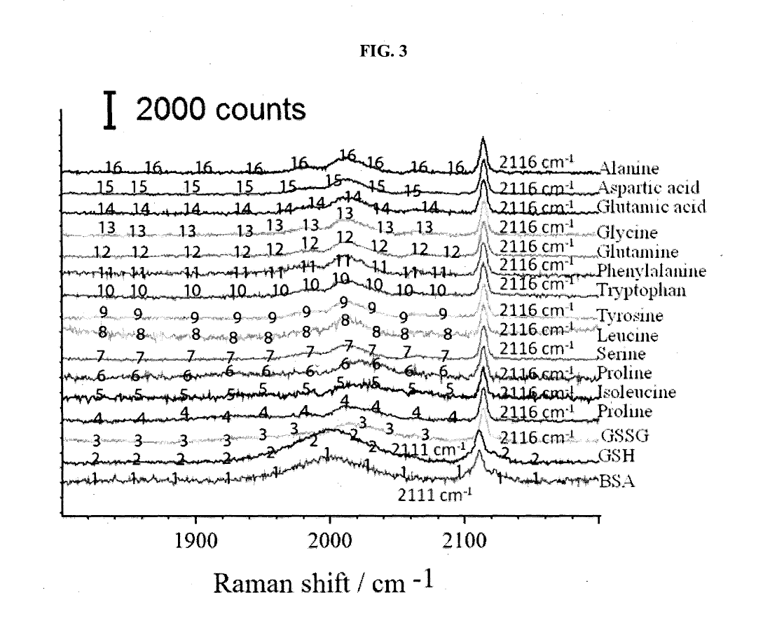

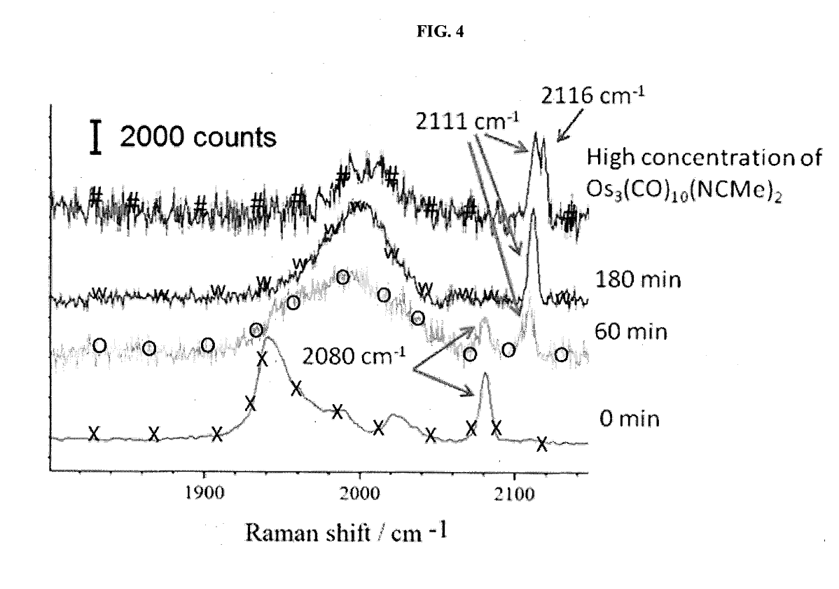

As shown in FIG. 4, in 1:1 ratio of Os₃(CO)₁₀(NCMe)₂ to GSH, the peak at 2,080 cm⁻¹ is firstly shifted to 2,111 cm⁻¹ which indicates that Os₃(CO)₁₀(NCMe)₂ reacts favorably with SH group than COOH group. In the concentration of Os₃(CO)₁₀(NCMe)₂ higher than that of GSH, two peaks at 2,111 cm⁻¹ and 2,116 cm⁻¹ were observed, suggesting that the excess Os₃(CO)₁₀(NCMe)₂ reacted with COOH group giving a peak at 2,116 cm⁻¹. Furthermore, GSH could be detected at least down to 20 μM (20 μM-10 mM) as shown in FIG. 5. Its SERS intensity is increased with the concentration of GSH. This results imply that Os₃(CO)₁₀(NCMe)₂ is sensitive and has a detection range that covers the physiological range 3-20 mM.

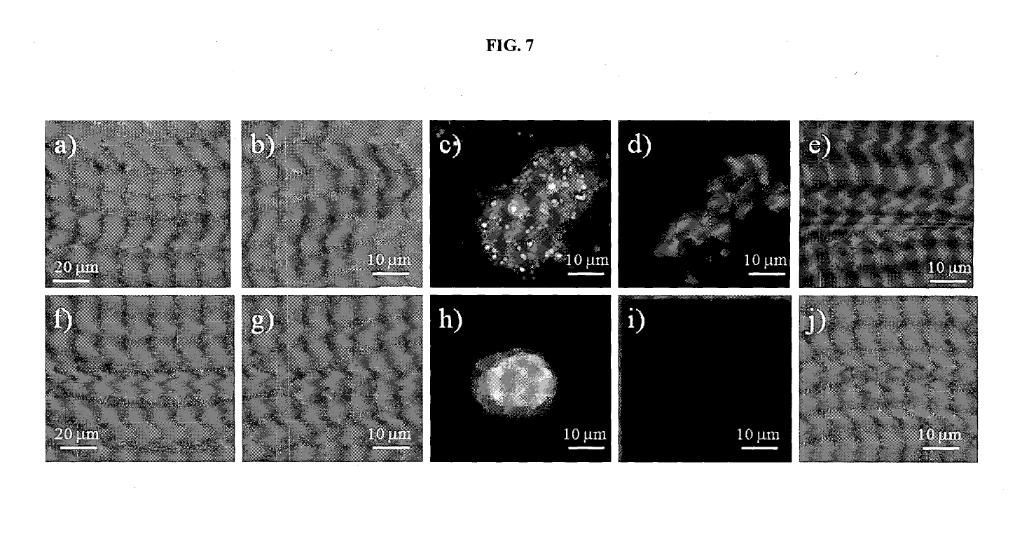

The application of Os₃(CO)₁₀(NCMe)₂ was further applied to detect SH containing molecules in living cells with the peak at 2,111 cm⁻¹ which was undertaken by Raman microscopy. Cells were incubated with Os₃(CO)₁₀(NCMe)₂ prior to introduction of gold nanoparticles into cells. The intracellular osmium carbonyl cluster signals can be enhanced by introducing gold nanoparticles. The gold nanoparticles were successfully introduced into cells as confirmed by the dark-field imaging as shown in FIG. 7C.

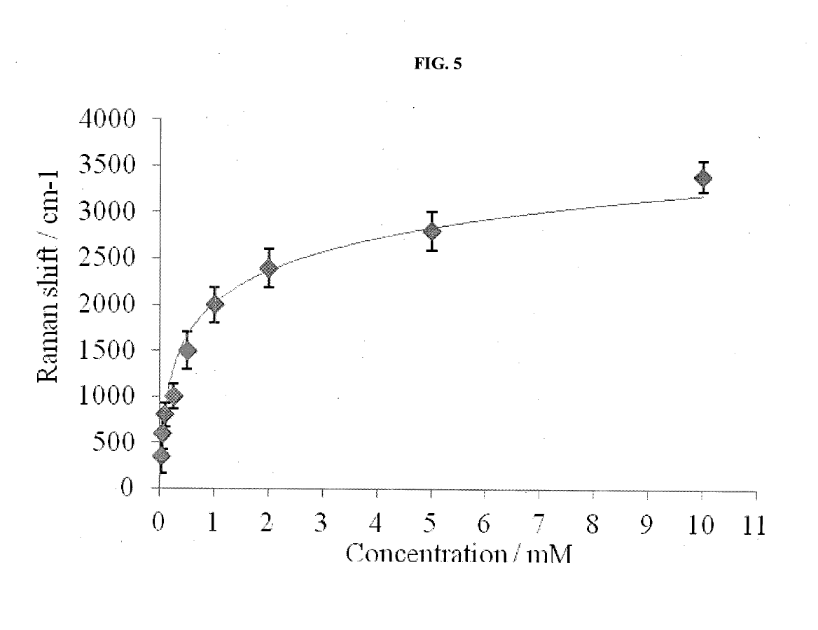

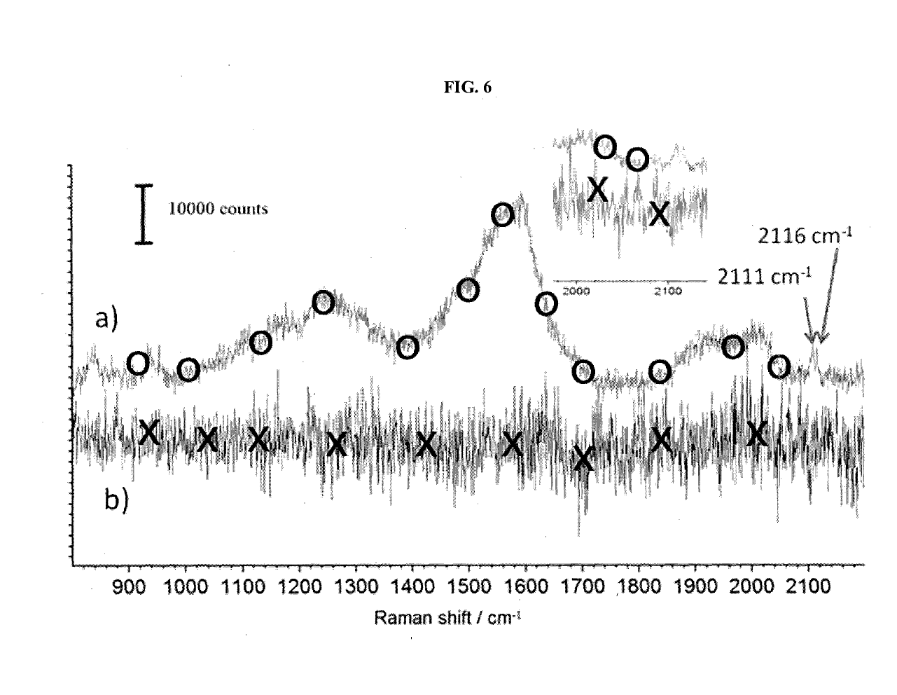

In contrast, cells without introduction of gold nanoparticles clearly showed the absence of light scattering's gold nanoparticles (FIG. 7H). In fact, cells that were incubated with Os₃(CO)₁₀(NCMe)₂ without introduction of gold nanoparticles showed no SERS signals of cellular biomolecules and CO of osmium carbonyl cluster (FIG. 6B and FIG. 7F-J). Whereas cells were incubated with Os₃(CO)₁₀(NCMe)₂ and subsequently with gold nanoparticles showed detectable signals of biomolecules and CO inside the cells (FIG. 6A and FIG. 7A-E). The results showed that Os₃(CO)₁₀(NCMe)₂ can easily penetrate cell membranes and make SH labeling. Considering the relative high cytosolic concentration of thiol containing molecules such as GSH and proteins in cells and the relationship between the cytosolic thiol level with many diseases, this probe may offer a new way to detect cytosolic thiol and in vitro thiol quantification with SERS.

### Example 7

### Results and Discussion for Probe with SERS Detection and Colorimetric Assay for Dual-Modal Sensing

A probe with SERS detection and colorimetric assay for dual-modal sensing was also developed in this study. Inclusion of both techniques would combine the high sensitivity of SERS with the convenient and visual appeal of a colorimetric for a more robust detection.

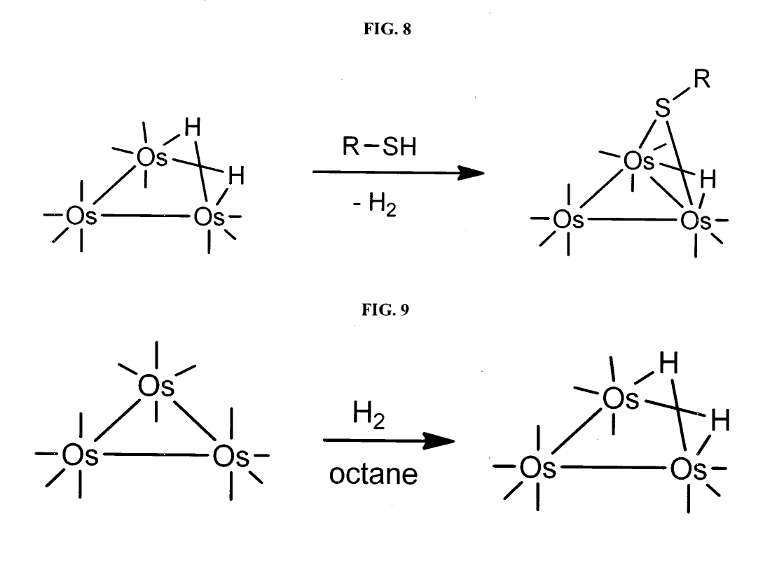

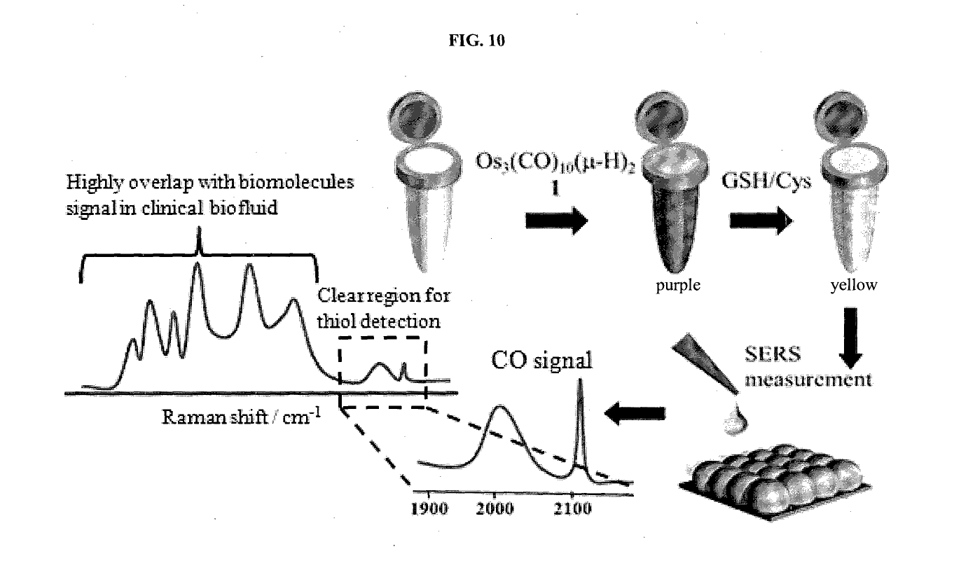

Os₃(CO)₁₀(μ-H)₂ is one of the reactive organometallic compounds with purple color due to its electronic unsaturation (46 electrons) (note: Unlike Os₃(CO)₁₀(μ-H)₂, Os₃(CO)₁₀(NCMe)₂ is in yellow before and after interaction with thiol. No color change but SERS change was observed.). It has a higher reactive towards thiol (—SH) functional group than other groups, Os₃(CO)₁₀(μ-H)₂ may react with thiol instantaneously to form a μ,η¹-thiolate bridged cluster (48 electrons) (FIG. 8). Formation of the thiolated cluster resulted in changes in the CO stretching vibration and a color change from purple to yellow. To the inventors' knowledge, this is the first report regarding use of SERS and colorimetric dual-modal probe for detection of thiol molecules.

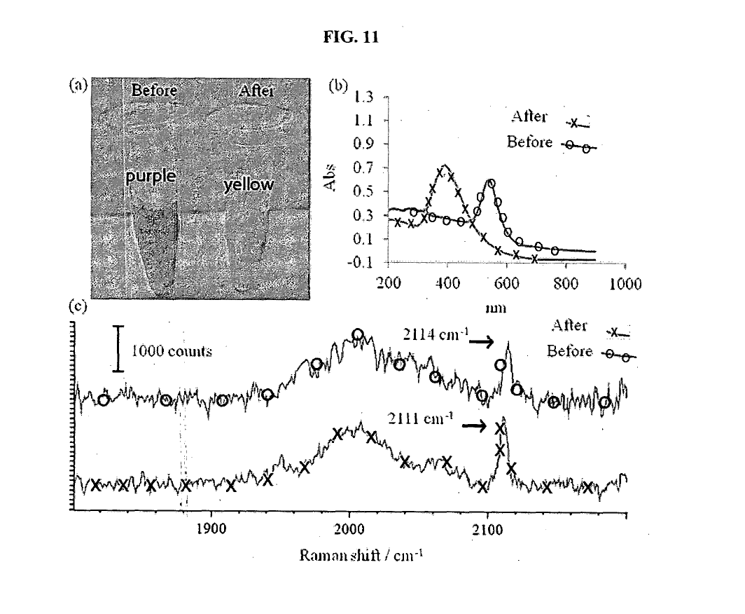

Os₃(CO)₁₀(μ-H)₂ may be synthesized easily from available starting materials (FIG. 9). In this study, reaction condition of Os₃(CO)₁₀(μ-H)₂ with thiol was optimized by using 1:1 ethanol-water to give maximum solubility and stability to Os₃(CO)₁₀(μ-H)₂ in the stock solution. The reaction of Os₃(CO)₁₀(μ-H)₂ with thiol is instantaneous, hence, a short incubation time of ten seconds was used throughout the experiments. Solution of Os₃(CO)₁₀(μ-H)₂ was freshly prepared prior to all experiments to ensure its stability. The reaction of Os₃(CO)₁₀(μ-H)₂ with cysteine was first investigated by UV-vis spectroscopy. Os₃(CO)₁₀(μ-H)₂ exhibits an absorption maximum at 530 nm (FIG. 11B). After 1:1 molar ratio mixing of Os₃(CO)₁₀(μ-H)₂ with cysteine, the absorption at 530 nm disappeared while a new peak at 390 nm developed. Such a huge shift in the absorption spectrum reflects the color change of the resultant solution from purple to yellow which is observable by the “naked-eye” (FIG. 11A).

The yellow solution was then examined by SERS. SERS substrate was used in the SERS study due to its capability in enhancing the CO stretching vibration significantly, hence, allowing monitor of the CO signals in extremely low sample volume (about 20 μL). Comparing the SERS spectrum of Os₃(CO)₁₀(μ-H)₂ (purple solution) with that of the yellow solution showed the shift of signal from 2114 cm⁻¹ to 2111 cm⁻¹ (FIG. 11C). The appearance of peak at 2111 cm⁻¹ is due to the formation of Os₃(CO)₁₀(μ-H)(μ-SR) species. The formation of Os₃(CO)₁₀(μ-H)(μ-SR) with cysteine was also supported by high-resolution mass spectrometry.

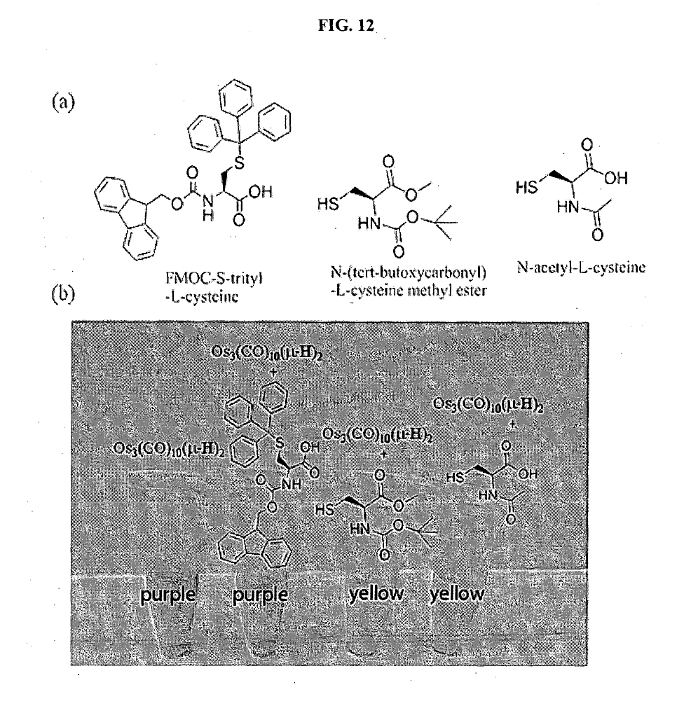

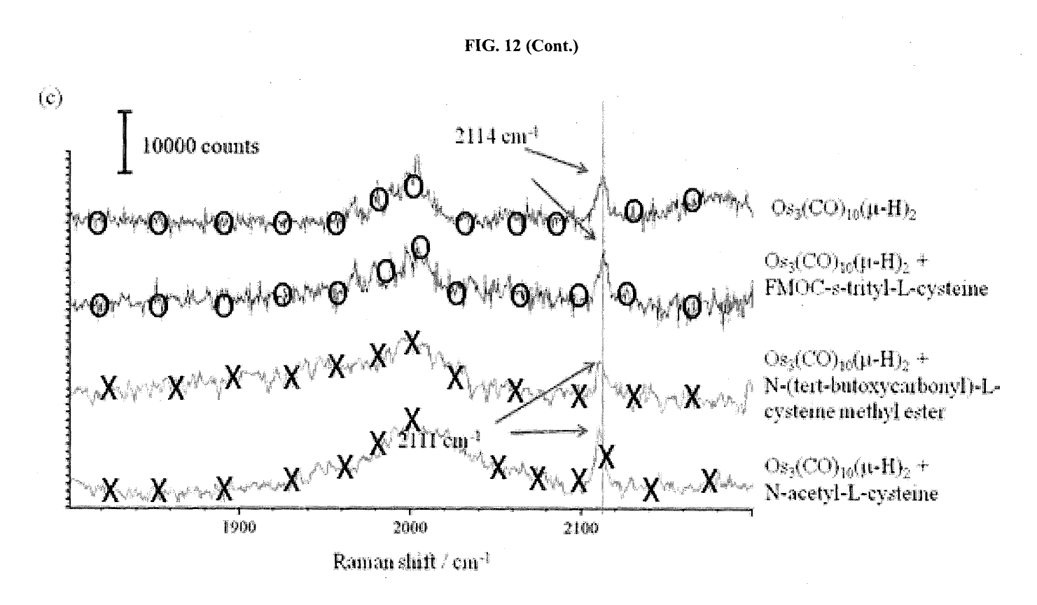

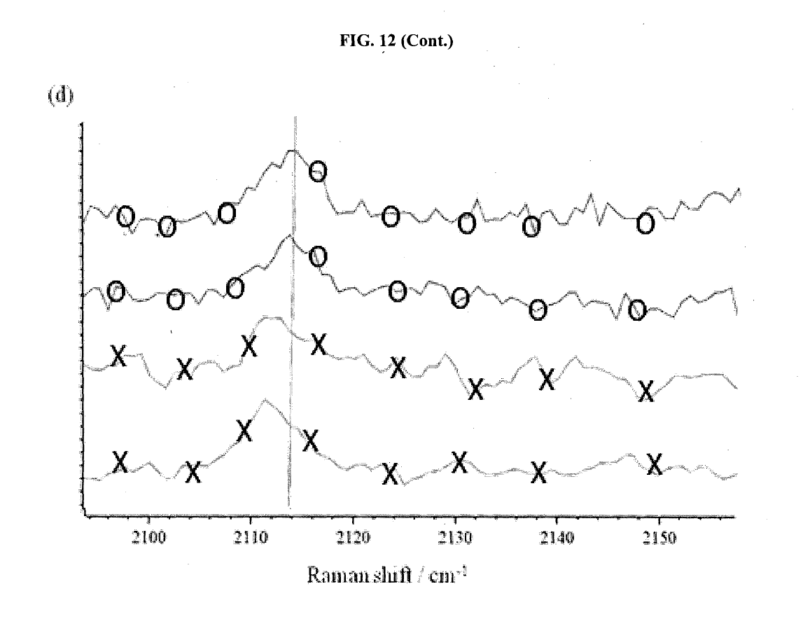

The thiol induced color and SERS changes were further verified with protected cysteines (FIG. 12). Incubating the solution of Os₃(CO)₁₀(μ-H)₂ with the carboxylic acid and amino groups protected cysteines also produced color change (yellow to purple) and SERS peak shift (2114 to 2111 cm⁻¹) similar to that of unprotected cysteine. On the other hand, the thiol protected cysteine does not exhibit color and SERS changes, suggesting the selective reactivity of Os₃(CO)₁₀(μ-H)₂ towards the thiol functionality. This also confirmed that the thiol functionality is responsible for the color and SERS spectrum changes.

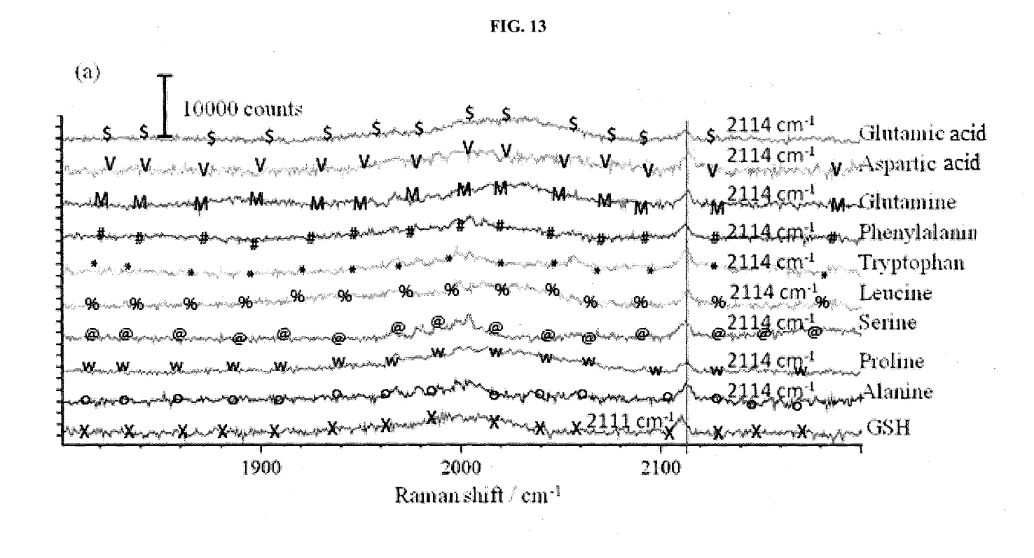

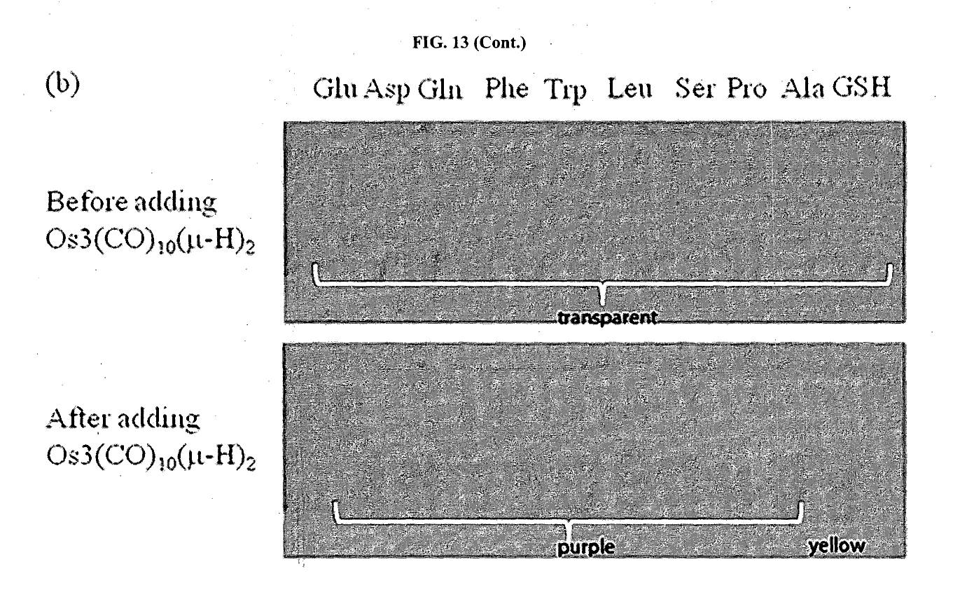

To further evaluate the selectivity of Os₃(CO)₁₀(μ-H)₂, it was treated with various amino acids such as GSH, and oxidized GSH (GSSG). No SERS peak changes were observed for all thiol-free amino acids, whereas the peak shift from 2114 to 2111 cm⁻¹ was observed for incubation with GSH (FIG. 13A). Furthermore, in the colorimetric detection, a distinct color change from purple to yellow was observed only for GSH but not for other thiol-free amino acids (FIG. 13B).

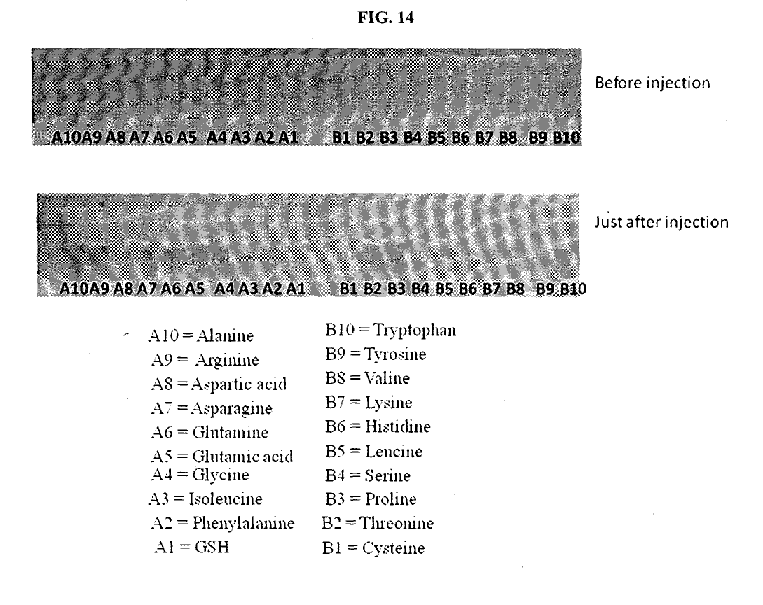

In a further colorimetric detection carried out, a distinct color change from purple to yellow was observed only for GSH but not for thiol-free amino acids (FIG. 14). This colorimetric observation is in line with the SERS results and it further confirms the high selectivity of Os₃(CO)₁₀(μ-H)₂ towards thiol functionality.

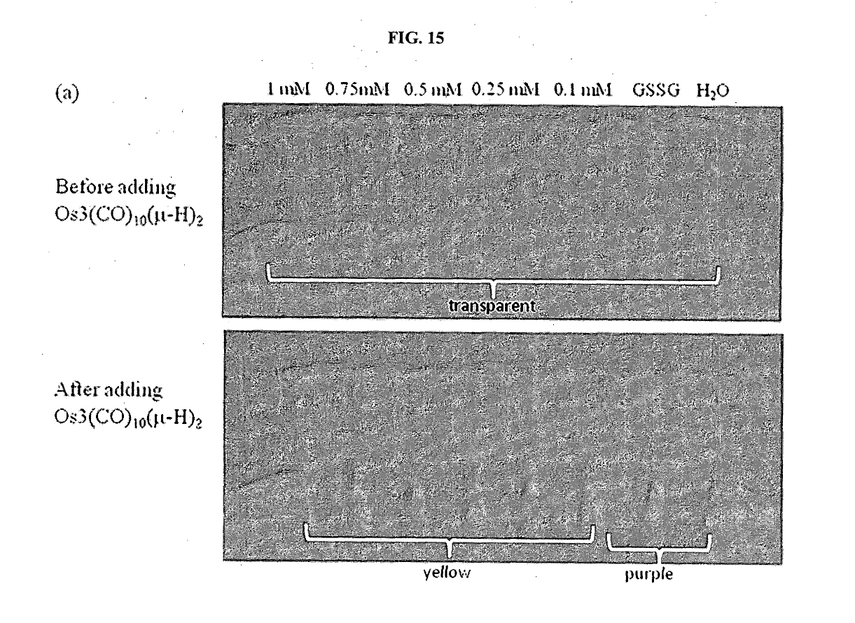

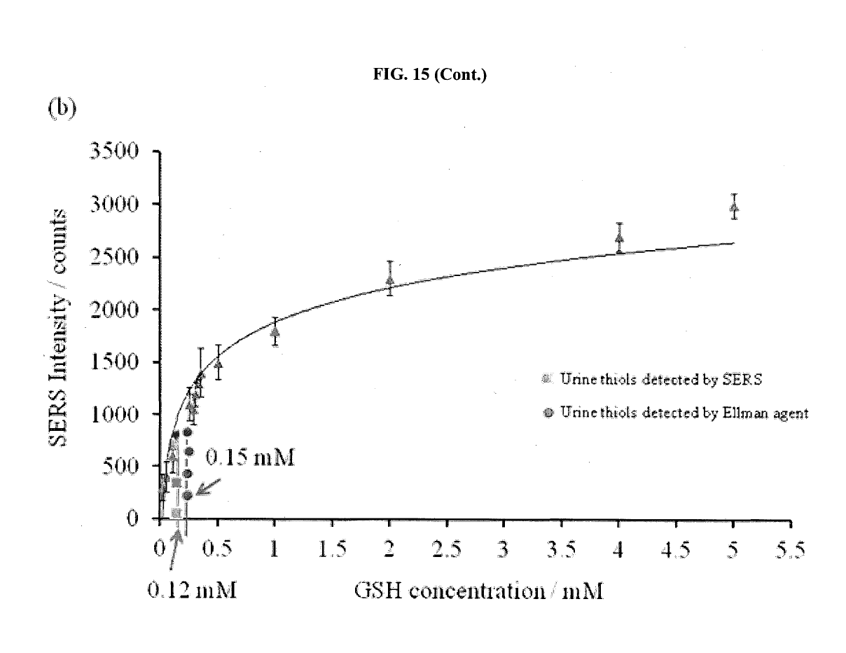

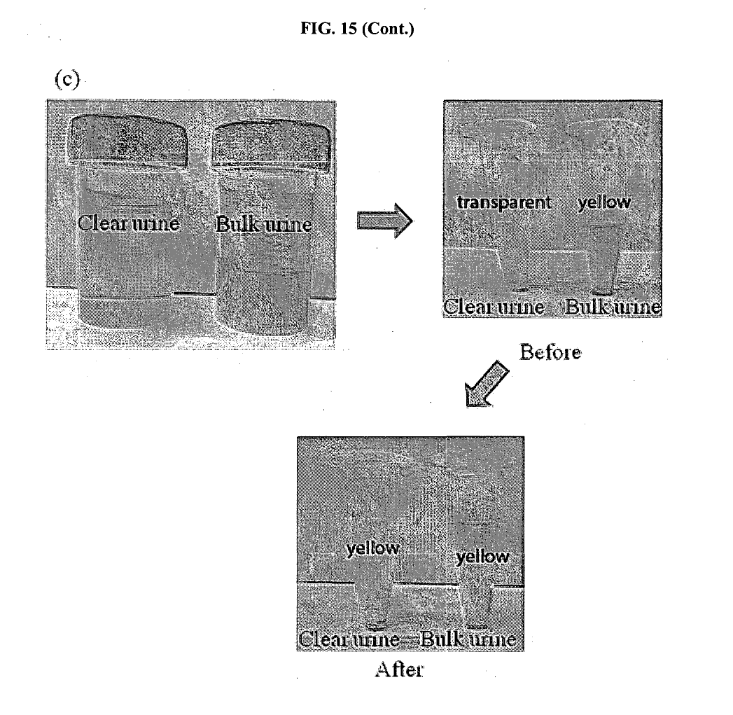

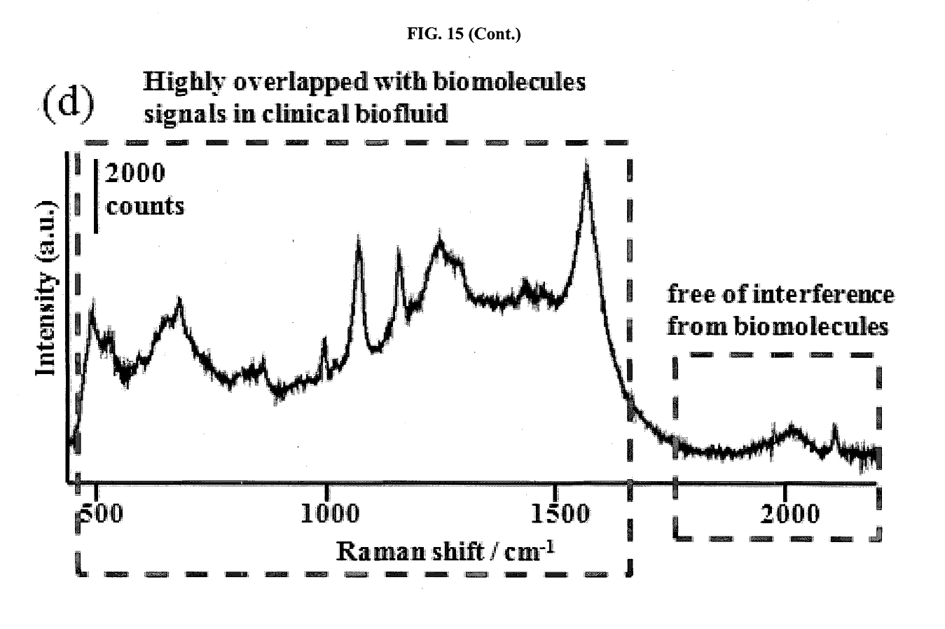

This approach was then applied to GSH quantification. Quantification of GSH was carried out with the addition of various concentrations of GSH into solutions of Os₃(CO)₁₀(μ-H)₂. This was carried out together with control experiments using water and oxidized GSH. Colorimetric detection by the eyes suggested that the color changes were apparent with increasing GSH concentrations and the visual detection limit may be as low as 0.1 mM (FIG. 15A). The control experiments exhibited no color change.

The SERS spectra of the solutions were also taken and the intensity of the peak at 2111 cm⁻¹ was plotted against concentration of GSH. The plot showed an increment with increasing concentration of GSH with the SERS intensity saturating at 5 mM. The detection limit by SERS was deduced to be 10 μM (FIG. 15B) and is lower than that by colorimetric detection, demonstrating that SERS is a more sensitive detection technique.

From this viewpoint, SERS can be considered as a complementary technique to colorimetric assay. Colorimetric assay offers the advantage of fast color development for rapid detection of GSH whereas SERS provides a highly sensitive detection for low concentration of GSH that cannot be detected by the naked-eye. As such, the dual modes can offer a choice of methods for different purposes.

However, it was also noted that bio-fluid sample, such as urine, with strong color may interfere with colorimetric detection, rendering ineffective. To verify this, urine sample was used in the study and the thiol concentration was quantified. The color development after adding Os₃(CO)₁₀(μ-H)₂ is clearly shown in clear urine sample, but overshadowed by the inherent color of bulk urine (FIG. 15C). Nevertheless, the thiol concentration in bulk urine was able to be quantified by SERS technique. The concentration was found to be 120 μM and it is in good agreement with the value determined by Ellman's reagent assay (150 μM).

The above shows that even though bio-fluid sample with strong color, such as urine, may interfere with the colorimetric detection, by complementing with use of SERS, a more accurate and sensitive detection method may be achieved.

This configuration has been used to determine the concentration of thiols in other human urine samples recently. Samples from two normal subjects and two patients with bladder cancer were analysed, and the values obtained were in good agreement with those determined by two separate commercial kits (TABLE 1). More work with clinical samples will be carried out.

TABLE 1
Thiol concentration comparison

| | Cystoscosy | Thiol concentration/μM | |
|---|---|---|---|
| | Findings | Our method | Thiol kit in the market |
| Urine sample 1 | Normal | 141 | 133 |
| Urine sample 2 | Normal | 90 | 84 |
| Urine sample 3 | Cancer | 50 | 46 |
| Urine sample 4 | Cancer | 41 | 37 |

In conclusion, a rapid and simple method, based on triosmium carbonyl cluster, for recognition and quantification of thiol containing biomolecules has been demonstrated. Os₃(CO)₁₀(NCMe)₂ and Os₃(CO)₁₀(μ-H)₂, which has a strong CO stretching vibrations in the mid-IR (1800-2200 cm⁻¹); a region which is relatively free of interference from absorbance of biomolecules, are reactive organometallic compounds with higher affinity towards thiol (SH) functionality as compared to that for carboxyl (COOH) and amide (NH₂) functionalities. Their reaction with biomolecules would significantly alter its metal carbonyl CO stretching vibrations and color. Use of Os₃(CO)₁₀(NCMe)₂ and Os₃(CO)₁₀(μ-H)₂ for thiol detection has been demonstrated.

The compounds provide a dual-mode of detection. Firstly, binding of thiol gave obvious color changes from purple to yellow which can be detected by the naked-eye. Secondly, it also showed an alteration in SERS spectrum in a region that is relatively free from interference from biomolecules. It provides a rapid and simple method, based on triosmium carbonyl cluster, for recognition and quantification of thiol containing biomolecules. This potentially opens up a new field where such SERS thiol probes are not available, and may find promising application in clinical diagnostics and real time biomolecules quantification in biocatalytic reaction at a low concentration and sample volume.

Embodiments disclosed herein present a highly sensitive, cost effective, fast, and simple to use method for detection of thiol functional groups in samples, which can translate into a biosensor that is easy to manufacture.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. Method of detecting one or more analytes having a thiol functional group, the method comprising
a. contacting one or more analytes with at least one metal carbonyl cluster compound; and
b. detecting changes in optical properties of the at least one metal carbonyl cluster compound as an indication of the presence of the one or more analytes having a thiol functional group.

2. Method according to claim 1, wherein the at least one metal carbonyl cluster compound has general formula (I)
M₃(CO)ₓL₁₂₋ₓ (I)
wherein
M at each occurrence denotes a metal selected from Group 6 to Group 11 of the Periodic Table of Elements;
x is an integer from 10 to 12; and
each L is independently a ligand having a dissociation constant that is at least 1×10⁻³ s⁻¹.

3. Method according to claim 2, wherein each L is independently a ligand having a dissociation constant that is higher than that of a thiol ligand.

4. Method according to claim 2, wherein each L is independently selected from the group consisting of —H, —NC, —CH₃, and —NC—(CH₂)ₙ—CH₃, wherein n is 0 or an integer from 1 to 10.

5. Method according to claim 2, wherein each L is independently selected from the group consisting of —H, and —NC—(CH₂)ₙ—CH₃, wherein n is 0 or an integer from 1 to 10.

6. Method according to claim 2, wherein M is independently selected from the group consisting of Cr, Mo, W, Mn, Tc, Re, Fe, Ru, Os, Co, Rh, Ir, Ni, Pd, Pt, Cu, Ag, and Au.

7. Method according to claim 2, wherein M is independently selected from the group consisting of Fe, Ru, and Os.

8. Method according to claim 2, wherein M is Os.

9. Method according to claim 2, wherein x is 10.

10. Method according to claim 1, wherein the at least one metal carbonyl cluster compound is selected from the group consisting of Os₃(CO)₁₀(μ-H)₂, Os₃(CO)₁₀(NC—CH₃)₂, and combinations thereof.

11. Method according to claim 1, wherein contacting one or more analytes with at least one metal carbonyl cluster compound comprises incubating the one or more analytes and the at least one metal carbonyl cluster compound with a metallic nanoparticle.

12. Method according to claim 11, wherein the metallic nanoparticle is coated with or consists of a metal selected from the group consisting of a noble metal, copper, aluminum, and alloys thereof.

13. Method according to claim 11, wherein the metallic nanoparticle is coated with or consists of gold, silver, or alloys thereof.

14. Method according to claim 1, wherein detecting changes in optical properties of the at least one metal carbonyl cluster compound is carried out with a naked eye and/or a spectrometer.

15. Method according to claim 14, wherein detecting changes in optical properties with a spectrometer comprises detecting changes in surface enhanced Raman signal from the at least one metal carbonyl cluster compound.

16. Method according to claim 15, wherein detecting changes in surface enhanced Raman signal from the at least one metal carbonyl cluster compound comprises detecting changes in pattern and/or intensity of surface enhanced Raman signal in the region of 1800 cm⁻¹ to 2200 cm⁻¹.

17. Method according to claim 1, wherein detecting one or more analytes having a thiol functional group further comprises determining amount of said analyte.

18. Method according to claim 17, wherein amount of the one or more analytes having a thiol functional group is correlated with surface enhanced Raman signal from the at least one metal carbonyl cluster compound.

19. Method according to claim 1, wherein the one or more analytes having a thiol functional group is contained in a sample and the detection is in vitro.

20. Method according to claim 1, wherein the one or more analytes having a thiol functional group is detected in a body fluid comprising the analyte.
